(19) United States
(12) Patent Application Publication
(10) Pub. No.: US 2026/0096845 A1
(43) Pub. Date: Apr. 9, 2026

(54) SURGICAL INSTRUMENT

(71) Applicant: Creo Medical Limited, Chepstow, South Wales (GB)

(72) Inventors: Louis TURNER, Chepstow (GB); Warren JONES, Chepstow (GB); Christopher Paul HANCOCK, Chepstow (GB); Duncan James Foster FITZSIMONS, St. Albans (GB)

(21) Appl. No.: 19/114,314

(22) PCT Filed: Sep. 7, 2023
(86) PCT No.: PCT/EP2023/074618

§ 371 Date: Mar. 21, 2025

(30) Foreign Application Priority Data
Sep. 23, 2022 (GB) 2213952.1

(51) Int. Cl. A61B 18/14; A61B 18/00; A61B 18/16; A61B 18/18

(52) U.S. Cl. CPC A61B 18/1445; A61B 18/16; A61B 18/1815; A61B 2018/00077; A61B 2018/00083; A61B 2018/00178; A61B 2018/00184; A61B 2018/00601; A61B 2018/00994; A61B 2018/162

(57) ABSTRACT

Various aspects provide an electrosurgical instrument featuring an instrument shaft with a second transmission line for RF EM energy conveyance. At the distal end of the shaft, a distal end assembly receives the RF EM energy and includes a pair of jaws that can move relative to each other, enabling the opening and closing of a gap between their opposing inner surfaces. The first jaw houses a second energy delivery structure designed to deliver RF EM energy for cutting through biological tissue. This structure includes a dielectric substrate with an exposed top surface at the gap between the jaws and an under surface on the opposite side of the substrate. Additionally, a second active conductive layer is positioned on the under surface of the dielectric substrate, specifically arranged on an exposed lip at the distal end of the first jaw, facilitating precise energy delivery during surgical procedures.

### FIELD OF THE INVENTION

The present invention relates to jaw configurations for surgical instruments, and particularly, although not exclusively, to jaw configurations for surgical instruments that may find use in electrosurgery. In some specific embodiments, the jaw configurations may be particularly suitable for sealing vessels, e.g. blood vessels.

### BACKGROUND

It is known to provide surgical and electrosurgical instruments with a pair of jaws at a distal end to enable gripping, sealing, or cutting of biological tissue. Such devices typically find application on the end of minimally invasive surgical laparoscopic tools but can equally find use in other clinical procedural areas such as gynaecology, endourology, gastrointestinal surgery, ENT procedures, etc. Depending on the context of use, these device can have differing physical construction, size, scale, and complexity.

There is scope to improve the configuration of jaws of such instruments to provide improved gripping of tissue and/or improved delivery of energy from the jaws to either gripped tissue or non-gripped tissue.

The present invention has been devised in light of the above considerations.

### SUMMARY OF THE INVENTION

At a general level, there is provided arrangements for surgical and electrosurgical instruments having related jaw configurations for improved gripping of tissue and/or improved delivery of energy to gripped tissue. For example, the tissue may be a vessel, e.g. a blood vessel, and the instruments may be used to seal the tissue, for example, tissue portions gripped between the jaws, using, for example, microwave frequency electromagnetic (EM) energy. Additionally or alternatively, the instrument may be used to cut or dissect tissue, for example, tissue outside the jaws, using, for example, radiofrequency (RF) EM energy.

According to a first aspect of the invention, there is provided an electrosurgical instrument comprising an instrument shaft comprising a second transmission line for conveying radiofrequency (RF) EM energy and a distal end assembly arranged at a distal end of the instrument shaft to receive the RF EM energy from the second transmission line. The distal end assembly comprises a pair of jaws that are movable relative to each other to open and close a gap between opposing inner surfaces thereof. A first jaw of the pair of jaws comprises a second energy delivery structure arranged to deliver the RF EM energy for cutting through biological tissue. The second energy delivery structure comprises a dielectric substrate having a top surface that is exposed at the gap between the opposing inner surfaces, and an under surface on an opposite side of the dielectric substrate from the top surface and a second active conductive layer on the under surface of the dielectric substrate. The second active conductive layer is arranged on an exposed lip at a distal end of the first jaw.

Providing an energy delivery structure including an active electrode on an exposed lip at a distal end of a jaw can provide a mechanism for fine tissue cutting and dissection to be performed. This may therefore enhance the functionality of the electrosurgical instrument. The conductive strip and layer may be free-standing, in that they may be handled separately of the dielectric substrate and bonded to the dielectric substrate. Alternatively, the conductive strip and layer may be a coating of the dielectric substrate, and may therefore not be able to exist independently of the substrate.

In some arrangements, the instrument shaft may comprise a first transmission line for conveying microwave electromagnetic (EM) energy. The first jaw may further comprise a first energy delivery structure arranged to receive the microwave EM energy from the first transmission line and to emit the microwave EM energy into the gap between the opposing inner surfaces. In an arrangement, the first energy delivery structure may comprise a ground conductive layer on the under surface of the dielectric substrate, the ground conductive layer being electrically isolated from the second active conductive layer, and an active conductive strip on the top surface of the dielectric substrate.

Providing an electrosurgical instrument having first and second energy delivery structures, and wherein the first and second energy delivery structures are mounted to the same dielectric substrate may provide increased ease of manufacture for an electrosurgical instrument, as well as enabling multiple forms of energy delivery as may be required for different forms of tissue treatment. In some embodiments, the first and second transmission lines are separate, for example, the first transmission line may be a coaxial cable and the second transmission line may be a twisted pair wire. In some other embodiments, the first and second transmission lines may share one or more of the same components, for example, the first transmission line may be a coaxial cable and the second transmission line may be a combination of one conductor (e.g. outer conductor) of the coaxial cable and a separate wire. Further, in some other embodiments, the first and second transmission lines may comprise the same element, for example, a coaxial cable.

In some arrangements, the first energy delivery structure may further comprise a ground conductive strip on the top surface of the dielectric substrate and electrically connected to the ground conductive layer.

The provision and location of a ground conductive strip on a top surface of the dielectric substrate may provide improved energy (e.g. microwave frequency EM energy) confinement within a region between the jaws of the electrosurgical device. For example, the ground conductive strip may flank the active conductive strip to improve lateral energy confinement. In some further examples, the ground conductive strip may surround a distal end of the active conductive strip to improve longitudinal energy confinement.

In some arrangements, the ground conductive strip may be electrically connected to the ground conductive layer via through holes formed in the dielectric substrate. Additionally or alternatively, in some arrangements, the ground conductive strip may be electrically connected to the ground conductive layer via a conductive strip on a side surface of the dielectric substrate. Each of these arrangements provides an implementation for connecting the ground conductive strip to the ground conductive layer. By providing the connection through or on the dielectric substrate, manufacturability of the electrosurgical instrument may also be improved by enabling the various connections, conductive layers, and conductive strips to be formed directly to the dielectric substrate.

In some arrangements, the first jaw may comprise an electrically conductive outer shell, the outer shell being electrically connected to the ground conductive layer. For example, the outer shell may be in direct physical contact with the ground conductive layer.

The electrically conductive outer shell, by being grounded, may thereby act as a return electrode for the first and/or second energy delivery structures. For example, the outer shell of the first jaw may act as a return electrode for RF EM energy (with the second active conductive layer as the active electrode) as well as part of a microwave frequency emitting structure or antenna (with the active conductive strip, ground conductive layer and/or the ground conductive strip). The configuration of the outer shell and the positioning of the active components with respect to the outer shell may also provide confinement of emitted energy with respect to the jaws, such as confinement within a region between the jaws, or may improve energy spread outwards from the region between the pair of jaws, as may be required for particular treatment processes and tissue types.

In some arrangements, the ground conductive strip may be electrically connected to the ground conductive layer via the electrically conductive outer shell. This provides an implementation for connecting the ground conductive strip to the ground conductive layer.

In some arrangements, the second jaw of the pair of jaws may comprise a grounded electrically conductive shell. This can therefore provide an additional return electrode or path for energy emitted from the first and/or second energy delivery structure. For example, the outer shell of the second jaw may act as a return electrode for RF EM energy (with the second active conductive layer as the active electrode) as well as part of a microwave frequency emitting structure or antenna (with the ground conductive layer, the outer shell of the first jaw and/or the ground conductive strip).

According to a second aspect of the invention, there is provided an electrosurgical instrument comprising an instrument shaft comprising a first transmission line for conveying microwave electromagnetic (EM) energy and a distal end assembly arranged at a distal end of the instrument shaft to receive the microwave EM energy from the first transmission line. The distal end assembly comprises a pair of jaws that are movable relative to each other to open and close a gap between opposing inner surfaces thereof. A first jaw of the pair of jaws comprises an energy delivery structure arranged to emit the microwave EM energy into the gap between the opposing inner surfaces. The energy delivery structure comprises a dielectric substrate having a top surface that is exposed at the gap between the opposing inner surfaces, and an under surface on an opposite side of the dielectric substrate from the top surface, a ground conductive layer on the under surface; and an active conductive strip on the top surface. The first jaw comprises an electrically conductive outer shell, the outer shell being electrically connected to the ground conductive layer.

An electrosurgical instrument having a jaw with an electrically conductive outer shell connected to the ground conductive layer (and hence being grounded) may provide confinement of emitted energy with respect to the jaws, such as confinement within a region between the jaws, or may improve energy spread outwards (e.g. laterally) from the region between the pair of jaws, as may be required for particular treatment processes and tissue types. Furthermore, the outer shell may act as a return electrode for the energy delivery structure(s). For example, the outer shell of the first jaw may act as a return electrode for RF EM energy (with the second active conductive layer as the active electrode) as well as part of a microwave frequency emitting structure or antenna (with the ground conductive layer and/or the ground conductive strip). For example, increasing a contact area between this return electrode and the tissue to be treated may provide improved energy delivery to the tissue.

In some arrangements, the jaw may comprise a longitudinally extending recessed groove on the inner surface of the jaw for receiving a blade for cutting through biological tissue.

In some arrangements, the dielectric substrate may be arranged on a top surface of the electrically conductive outer shell, the top surface of the electrically conductive outer shell being a surface of the electrically conductive outer shell closest to the inner surface of the first jaw. The recessed groove for receiving the blade extends from the inner surface of the jaw through the dielectric substrate and into the electrically conductive shell. The conductive shell may therefore act, in effect, as an electromagnetic shield for the energy delivery structure (e.g. for microwave frequency EM energy), and may direct energy upwards towards a region between the pair of jaws.

In some arrangements, the dielectric substrate may be received in a second recessed groove on a top surface of the electrically conductive outer shell, the top surface of the electrically conductive outer shell being a surface of the electrically conductive outer shell closest to the inner surface of the first jaw. The recessed groove for receiving the blade may extend from the inner surface of the jaw into the electrically conductive shell and adjacent to the recessed groove for receiving the blade.

In this way, the electrically conductive shell may also influence (e.g. increase) the lateral distribution of energy (e.g. microwave frequency EM energy) from the active conductive strip.

In some arrangements, the second recessed groove may be open to a side surface of the first jaw, such that the dielectric substrate is exposed at the side surface of the jaw (e.g. an outer side surface). This may direct energy emitted from the active conductive strip outwardly in a lateral direction of the jaw.

In some arrangements, the second recessed groove is within the electrically conductive shell. In other words, the electrically conductive shell may surround the under surface and the side surface(s) (e.g. all surfaces other than the top surface facing the gap) of the dielectric substrate. This may direct energy emitted from the active conductive strip in a focussed manner towards a region between the pair of jaws.

In some arrangements, the second recessed groove may be a U-shaped groove that flanks the recessed groove for receiving the blade and surrounds its distal end. This may thereby improve the lateral confinement or distribution of energy from the energy delivery structure.

In some arrangements, the second jaw of the pair of jaws may comprise a grounded electrically conductive shell. This can therefore provide an additional return electrode or path for energy emitted from the first and/or second energy delivery structure. For example, the outer shell of the second jaw may act as a return electrode for RF EM energy (with the second active conductive layer as the active electrode) as well as part of a microwave frequency emitting structure or antenna (with the ground conductive layer, the outer shell of the first jaw and/or the ground conductive strip).

In some arrangements, the instrument may further comprise a blade for cutting through biological tissue, the blade being slidably mounted within the recessed groove. The blade may be slidable in a longitudinal direction between a retracted position in which the blade is stowed to avoid unintentionally cutting tissue in the gap (e.g. when stowed, the blade may lie proximal to the pair of jaws) and an extended position for cutting tissue in the gap (e.g. in this position the blade may lie within a region between the pair of jaws), or may be slidable in a lateral direction between a retracted position in which it lies beneath the inner surface of the first jaw and an extended position in which it lies within a region between the pair of jaws. The blade may comprise a rigid element with a sharp edge adapted to slice biological tissue. The blade may thereby provide additional functionality to the electrosurgical instrument by enabling a physical cut of tissue gripped between the jaws of the instrument. The blade may perform a “cold cut”, that is, cutting via a sharp blade rather than via EM energy (e.g. RF EM energy).

In some arrangements, the instrument shaft may comprise a second transmission line for conveying radiofrequency (RF) EM energy. The pair of jaws may comprise a second energy delivery structure arranged to deliver the RF EM energy for cutting through biological tissue. The second energy delivery structure may comprise a second active conductive layer on the under surface of the dielectric substrate, the second active conductive layer being arranged on an exposed lip at a distal end of the first jaw and being electrically isolated from the ground conductive layer. This additional energy delivery system may provide additional functionality, such as fine tissue cutting at a distal end of the instrument.

According to a third aspect of the invention, there is provided an electrosurgical instrument comprising an instrument shaft arranged to convey radiofrequency (RF) EM energy and a distal end assembly arranged at a distal end of the instrument shaft to receive the RF EM energy from the instrument shaft. The distal end assembly comprises a pair of jaws that are movable relative to each other to open and close a gap between opposing inner surfaces thereof. A second jaw of the pair of jaws comprises an electrically conductive outer shell and an active electrode arranged on an outer surface of the jaw, the active electrode being for delivering RF energy to biological tissue, the active electrode being electrically isolated from the electrically conductive outer shell. The electrically conductive outer shell is grounded to form a return electrode for the RF energy.

In an embodiment, the active electrode may be positioned at a distal end, for example, on a distal end face, of the second jaw.

The active electrode on the outer surface of the second pair of jaws may provide a functionality as fine tissue cutting at a distal end of the instrument.

In some examples, the electrode may be comprised within a dielectric plug, the dielectric plug forming part of the outer surface of the jaw and being arranged to electrically isolate the active electrode from the electrically conductive outer shell. In other words, the outer shell may have a hole in the outer surface of the jaw that is filled by the dielectric plug containing the electrode. This may provide a particular implementation of the energy delivery structure. In an embodiment, the active electrode may be substantially circular, and the plug may be an annulus around the active electrode.

In some examples, the active electrode may be substantially flush with the outer surface of the jaw. This may reduce catching or snagging of the instrument on tissue, particularly when working in a confined space.

In some arrangements, the electrosurgical instrument of the first, second, or third aspects may be arranged to receive and emit electromagnetic energy (e.g. RF energy and/or microwave frequency energy) from an electrosurgical generator. Such electrosurgical generators are known, e.g. as disclosed in WO 2012/076844.

According to a fourth aspect of the invention, there is provided a surgical instrument comprising an instrument shaft and a distal end assembly arranged at a distal end of the instrument shaft. The distal end assembly comprises a pair of jaws that are movable relative to each other to open and close a gap between opposing inner surfaces thereof, the jaws being connected at a pivot point. A second jaw of the pair of jaws comprises a strut connected to the pivot point, a rigid jaw shell forming an outer surface of the second jaw; and a hinge element connecting the strut to the rigid jaw shell such that the rigid jaw shell can pivot on (or with respect to) the strut.

This can provide a jaw that produces more constant pressure along a length of the jaw by enabling a jaw of the pair of jaws to flex or pivot to distribute the pressure of the jaws more evenly.

In some arrangements, the strut, the hinge element, and the rigid jaw shell may be a unitary component of the surgical instrument. The hinge element in this scenario may be termed a living hinge, and/or may be a thinned or corrugated portion of the unitary component that thereby has an increased flexibility compared to other parts of the unitary component. Other arrangements for the hinge element are also possible. For example, where the hinge element is not unitary with the strut and/or rigid jaw shell, the hinge may nevertheless have an increased flexibility compared to the strut and/or rigid jaw shell such that the rigid jaw shell can pivot on the strut.

In some arrangements, the surgical instrument may further comprise a flexible body forming an inner surface of the second jaw. The flexible body may further improve pressure distribution, and may enable the inner surface of the jaw to at least partially mould, adapt, or conform to the gripped tissue.

In an embodiment, the flexible body may be further positioned in-between the strut and the rigid jaw shell. The flexible body may further be positioned in-between the hinge element and the rigid jaw shell. In this way, the flexible body may dampen or soften the pivoting of the rigid jaw shell with respect to the strut such that the evening of pressure between the jaws is more controllable and predictable.

In an embodiment, the strut comprises a proximal portion which projects proximally from the rigid jaw shell for connecting the second jaw to the pivot point. In an embodiment, the strut comprises a distal portion which extends into the rigid jaw shell. For example, the hinge element may be positioned about halfway along a length of the rigid jaw shell, and the strut may extend into the rigid jaw shell to meet the hinge element. In this way, a second pivot point defined by the hinge element may be substantially midway along the length of the rigid jaw shell.

According to a fifth aspect of the invention, there is provided a surgical instrument comprising an instrument shaft and a distal end assembly arranged at a distal end of the instrument shaft. The distal end assembly comprises a pair of jaws that are movable relative to each other to open and close a gap between opposing inner surfaces thereof. A second jaw of the pair of jaws comprises a flexible body forming an inner surface of the second jaw; and a rigid jaw shell attached to the flexible body and forming an outer surface of the first jaw. The inner surface of the second jaw has a distal contact portion and a proximal contact portion, the distal contact portion being configured to contact a first jaw of the pair of jaws before the proximal contact portion when the pair of jaws are moved towards each other and into contact.

In some arrangements, a thickness of the flexible body may increase along a length of the second jaw in a distal direction, such that the distal contact portion is configured to contact the first jaw (e.g. an inner surface thereof) before the proximal contact portion contacts the first jaw (e.g. an inner surface thereof) when the pair of jaws are moved towards each other and into contact, e.g. when the jaws are closed.

This increased thickness of the flexible body may enable the distal end of the jaw to contact before the proximal end of the jaw, and to flex as the jaws are further brought into contact to maintain a substantially even pressure along a length of the jaw,

In some arrangements, the rigid jaw shell may be curved towards the opposite jaw along a length of the first jaw in a distal direction, such that the distal contact portion is configured to contact the first jaw (e.g. an inner surface thereof) before the proximal contact portion contacts the first jaw (e.g. an inner surface thereof) when the pair of jaws are moved towards each other and into contact, e.g. when the jaws are closed. This bend of the rigid shell may enable the distal end of the jaw to contact before the proximal end of the jaw, and to flex as the jaws are further brought into contact to maintain a substantially even pressure along a length of the jaw. In other words, the rigid jaw shell may be shaped to resemble an arm of a tweezer such that a distal portion of the rigid jaw shell may deflect towards the first jaw as it progresses distally along its length. For example, an amount of curvature along the length may be constant or may increase in a distal direction. In an embodiment, the rigid jaw shell may be made from a resilient material, such as metal, e.g. steel or sprung steel.

An embodiment may employ a combination of (i) increased thickness of the flexible portion and (ii) a curved rigid jaw shell to enable the distal contact portion of the second jaw to contact the inner surface of the first jaw before the proximal contact portion of the second jaw contacts the inner surface of the first jaw.

The invention includes the combination of the aspects and preferred features described except where such a combination is clearly impermissible or expressly avoided. For example, in an embodiment, the surgical instruments of the fourth and fifth aspects are electrosurgical instruments having some or all of the electrosurgical features of the first, second and third aspects. Additionally or alternatively, in an embodiment, the electrosurgical instruments of the first to third aspects include some or all of the surgical instrument features of the fourth and fifth aspects.

In addition, the terms “first jaw” and “second jaw” are intended to identify a jaw of the pair of jaws rather than to be limiting. Thus, features described as being in the “first jaw” may instead be in the “second jaw” and vice versa.

### SUMMARY OF THE FIGURES

Embodiments and experiments illustrating the principles of the invention will now be discussed with reference to the accompanying figures in which:

FIG. 1 illustrates a distal end assembly according to an aspect of the present invention;

FIG. 2 illustrates a jaw of the distal end assembly of FIG. 1;

FIG. 3 illustrates a cross-sectional view of the jaws of the distal end assembly of FIG. 1;

FIG. 4 illustrates a cross-sectional view of a jaw of the distal end assembly of FIG. 1;

FIG. 5 illustrates a cross-sectional view of a second arrangement for a jaw of the distal end assembly of FIG. 1;

FIG. 6 illustrates a cross-sectional view of a third arrangement for a jaw of the distal end assembly of FIG. 1;

FIG. 7 illustrates a cross-sectional view of a fourth arrangement for a jaw of the distal end assembly of FIG. 1;

FIG. 8 illustrates an energy delivery structure for the distal end assembly of FIG. 1;

FIG. 9 illustrates a second view of the energy delivery structure of FIG. 8;

FIG. 10 illustrates a third view of the energy delivery structure of FIG. 8;

FIG. 11 illustrates a fourth view of the energy delivery structure of FIG. 8;

FIG. 12 illustrates a second energy delivery structure for the distal end assembly of FIG. 1;

FIG. 13 illustrates a second view of the energy delivery structure of FIG. 12;

FIG. 14 illustrates a third energy delivery structure for the distal end assembly of FIG. 1;

FIG. 15 illustrates a detail of the distal tip of the distal end assembly of FIG. 1;

FIG. 16 illustrates a second view of the distal tip of FIG. 15;

FIG. 17 illustrates a distal end assembly according to an aspect of the present invention;

FIG. 18 illustrates a second view of the distal assembly of FIG. 17;

FIG. 19 illustrates a third view of the distal assembly of FIG. 17;

FIG. 20 illustrates part of a jaw of a surgical instrument according to an aspect of the present invention;

FIG. 21 illustrates a second view of the jaw of FIG. 20;

FIG. 22 illustrates a third view of the jaw of FIG. 20;

FIG. 23 illustrates part of a jaw of a surgical instrument according to an aspect of the present invention;

FIG. 24 illustrates part of a jaw of a surgical instrument according to an aspect of the present invention;

FIG. 25 illustrates a second view of the jaw of FIG. 24; and

FIGS. 26 and 27 illustrate a working principle of the jaw of FIG. 24.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

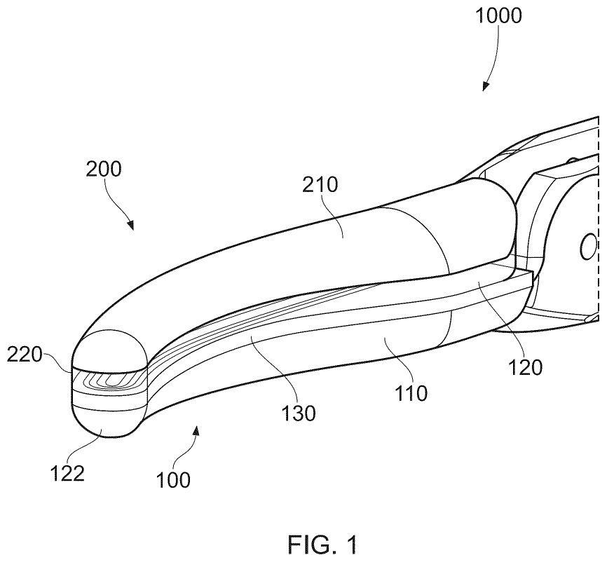

FIG. 1 illustrates a distal end assembly 1000 of an electrosurgical instrument of the present invention. The distal end assembly comprises a first jaw 100 and a second jaw 200, the first jaw 100 and second jaw 200 being movable relative to each other to open and close a gap between opposing inner surfaces thereof.

The first jaw 100 comprises a conductive shell (or conductive rigid jaw shell) 110 and an energy delivery structure. The energy delivery structure comprises a dielectric substrate 130. In this embodiment, an insulating coating (or insulating flexible body) 120 is provided on an inner surface of the first jaw 100. In other examples, this coating 120 may be omitted. The coating may provide, for example, improved tissue gripping and/or reduced tissue sticking to the inner surface of the jaw 100. The first jaw 100 of this embodiment further comprises a flexible tip 122 that may enable, for example, improved movement past biological tissue by reducing friction between the jaw 100 and the tissue.

The second jaw 200 comprises a conductive outer shell (or conductive rigid jaw shell) 210 and a flexible element (or body) 220 on an inner surface of the jaw. The conductive outer shell 210 may be grounded to provide a further return electrode for emitted energy from the energy delivery structures of the distal end assembly 1000.

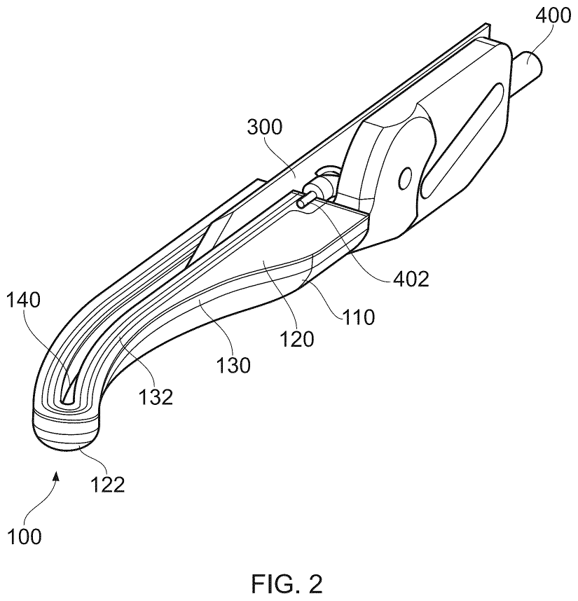

FIG. 2 illustrates the first jaw 100. The illustrated first jaw 100 includes an active conductive strip 132 on the dielectric substrate 130. The active conductive strip 132 is connected to a power cable, which in this embodiment is a coaxial power feed cable 400 having an inner conductor and an outer conductor separated by a dielectric layer. The coaxial cable 400 is connected to the active conductive strip 132 via a terminal 402. For example, the inner conductor of the coaxial cable 400 may be soldered to the active conductive strip.

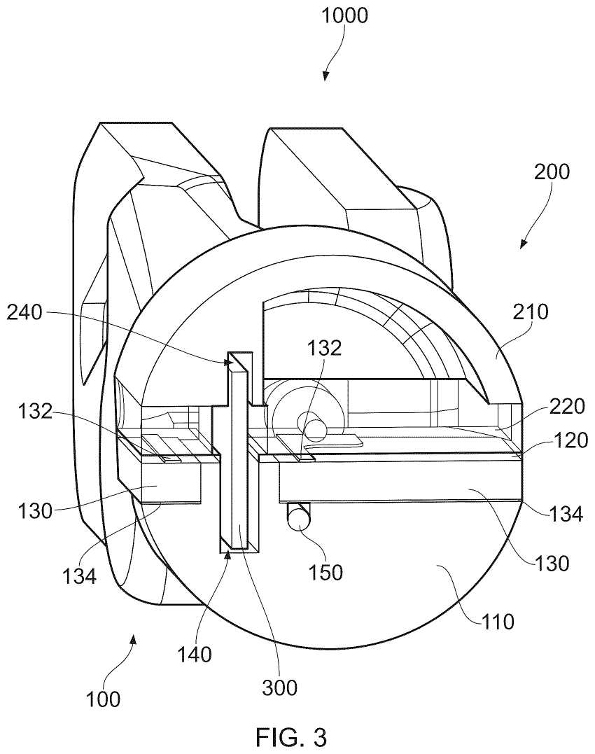

The first jaw 100 further comprises a blade 300, which is slidably mounted in a groove 140. This blade 300 is a blade for cutting through biological tissue. The blade 300 of this embodiment is slidable in a longitudinal direction along the groove 140 to cut tissue in the region between the first jaw 100 and the second jaw 200. This groove 140 and blade 300 arrangement can also be seen in FIG. 3, which illustrates a cross-section through the first jaw 100 and second jaw 200. In this embodiment, the second jaw 200 includes a corresponding groove 240 to the groove 140 of the first jaw 100.

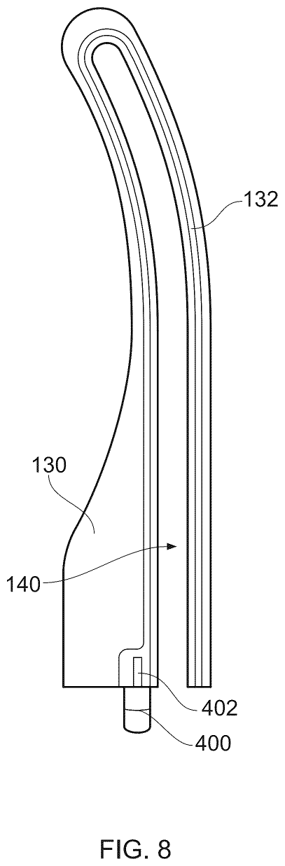

FIG. 3 further illustrates components of the energy delivery structures of the first jaw 100, which are shown in greater detail in FIGS. 8 to 11. The ground conductive layer 134 on the under surface or lower surface of the dielectric substrate can be seen. The ground conductive layer 134 is arranged in physical and electrical contact with the electrically conductive shell 110. The first jaw 100 of this embodiment further includes a feed cable 150 for providing energy to the second energy delivery structure of the first jaw 100.

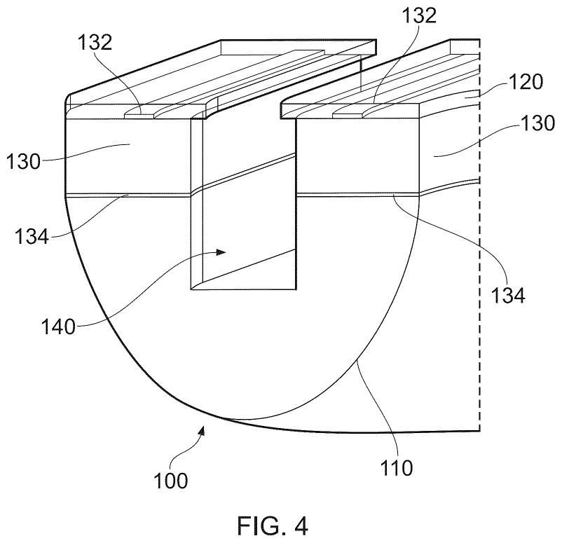

FIGS. 4 to 7 illustrate alternative relative arrangements for the groove 140 with respect to the dielectric substrate 130, the ground conductive layer 134 and the active conductive strip 132.

In the embodiment of FIG. 4, the dielectric substrate 130 is arranged on a top surface of the conductive shell 110. The groove 140 extends downwards through each of the dielectric substrate 130 and the conductive shell 110. In this embodiment, the ground conductive layer 134 and the grounded conductive shell 110 are arranged entirely below the active conductive strip 132. Thus, the ground conductive layer 134 and the grounded conductive shell 110 serve as an electromagnetic shield to direct energy emitted from the active conductive strip 132 in an upward direction towards the region between the pair of jaws 100,200.

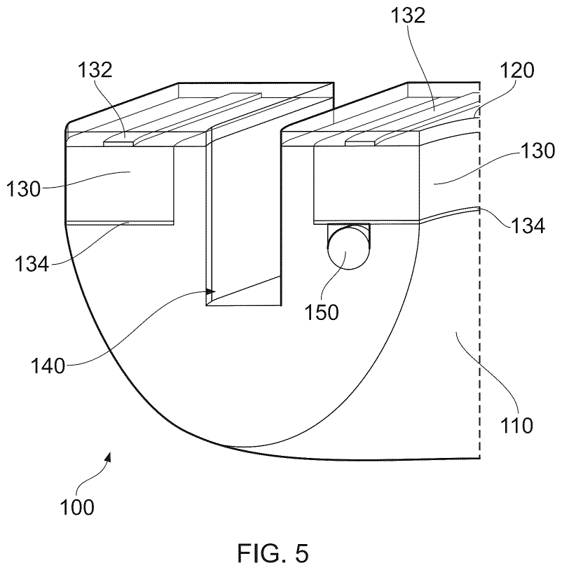

In the embodiment of FIG. 5, the conductive shell 110 extends upwards past an inner surface of the dielectric substrate 130 to form the wall of the groove 140. In this way, the ground plane provided by the conductive shell 110 and the ground conductive layer 134 extends centrally of (e.g. at the centre and on the side of) the active conductive strip 132 as well as beneath the active conductive strip 132. This may direct energy emitted from the active conductive strip outwardly of the jaws 100, 200 in a lateral direction (i.e. away from the groove 140 and blade 300).

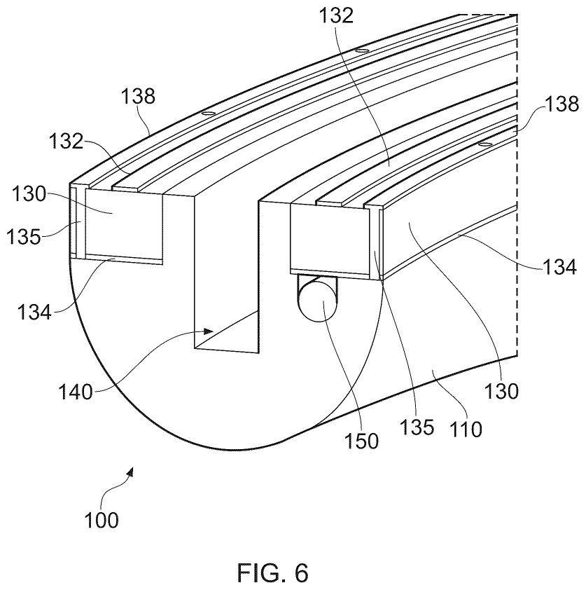

In the embodiment of FIG. 6, the energy delivery structure further comprises a ground conductive strip 138 on a top surface of the dielectric substrate 130. In this embodiment, the ground conductive strip 138 is connected to the ground conductive layer 134 via one or more through holes 135 filled with conductive material. Where multiple through holes 135 are present, they may be evenly distributed around the dielectric substrate, or located only in a proximal region of the jaw. In other examples, the ground conductive strip 138 may be connected to the ground conductive layer 134 via a coating on the side of the dielectric substrate 130. The presence of this ground conductive strip 138 may further confine energy emitted from the active conductive strip 132 such that it is directed upwards of the active conductive strip in a narrow band towards the region between the pair of jaws 100, 200, for example, and not in any lateral direction.

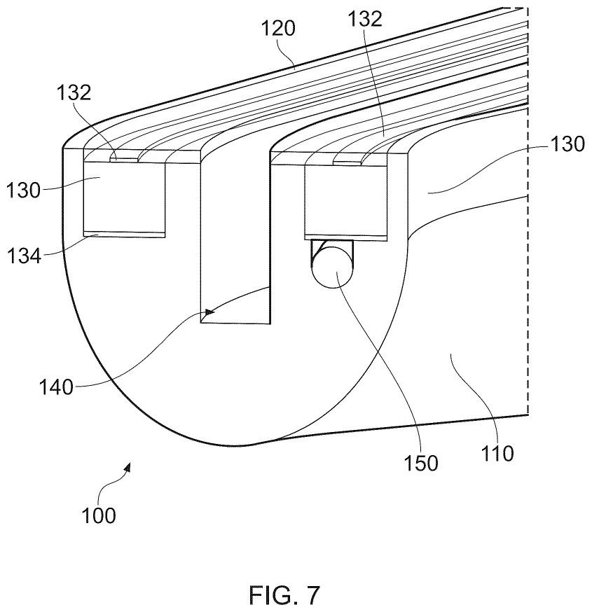

A similar effect may be achieved by the embodiment of FIG. 7, wherein the conductive shell 110 extends upwardly on an outer side and an inner side of the dielectric substrate 130. In other words, the dielectric substrate sits within an enclosed trench on a top surface of the conductive shell 110. As with the ground conductive strip 138, this arrangement may deliver energy from the active conductive strip in a narrow band directed into the region between the pair of jaws 100, 200, for example, and not in any lateral direction.

The arrangements of FIGS. 4 to 7 each illustrate a symmetrical arrangement, wherein the configuration of the dielectric substrate 130 and conductive shell 110 are similar on both sides of the groove 140. However, in other embodiments, the configurations of FIGS. 4 to 7 may be combined. For example, an arrangement as in FIG. 4 on one side of the groove 140 may be combined with an arrangement as in FIG. 5, 6 or 7 on the other side of the groove 140. An arrangement as in FIG. 5 on one side of the groove 140 may be combined with an arrangement as in FIG. 6 or 7 on the other side of the groove 140. An arrangement as in FIG. 6 on one side of the groove 140 may be combined with an arrangement as in FIG. 7 on the other side of the groove 140. These configurations may be selected as required to deliver the particular energy distribution pattern wanted from the energy delivery structure.

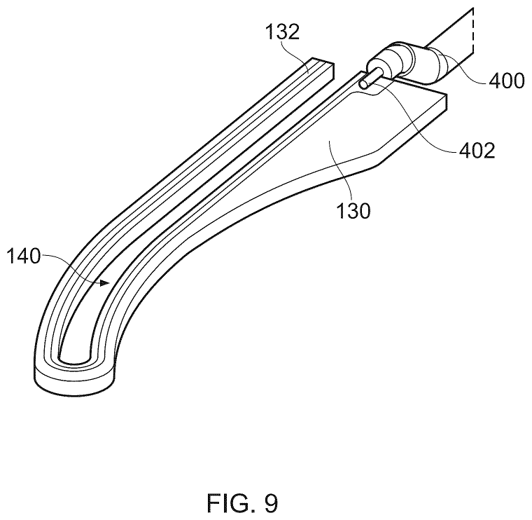

FIGS. 8 to 11 illustrate the energy delivery structure in isolation from the first jaw 100. FIGS. 8 and 9 show the top surface of the dielectric substrate 130, illustrating the arrangement of the active conductive strip as a U-shaped strip flanking the groove 140 and surrounding its distal end.

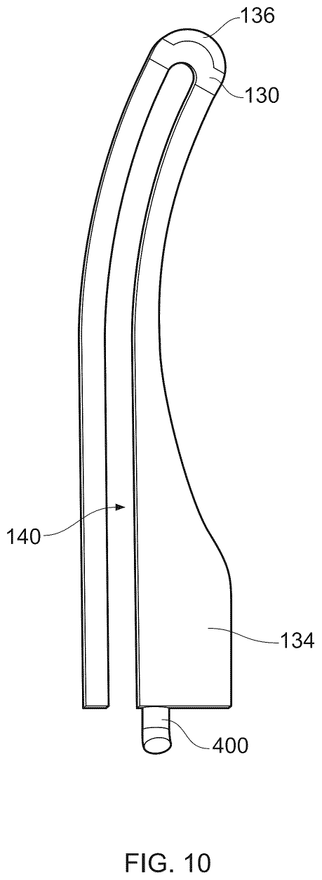

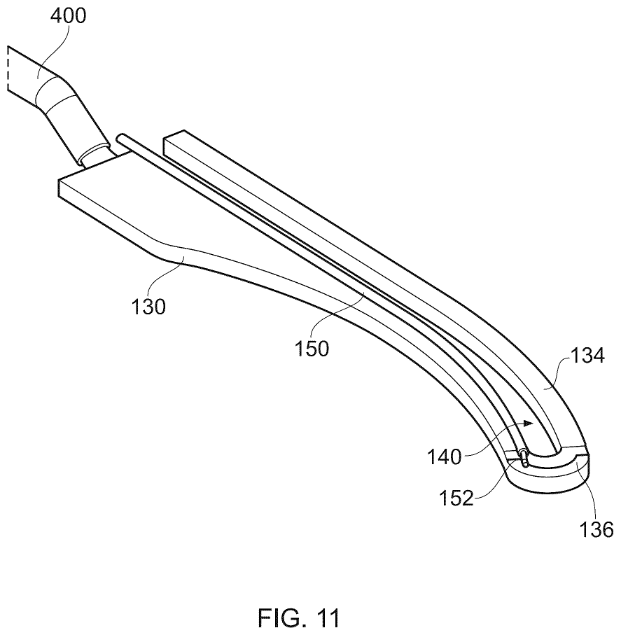

FIGS. 10 and 11 illustrate the under surface of the dielectric substrate 130. These figures illustrate the ground conductive layer 134 on the under surface. The figures further illustrate the active conductive layer (e.g. second active conductive layer) 136 arranged at a distal end of the dielectric substrate 136. The ground conductive layer 134 flanks the groove 140 through the dielectric substrate 130. The active conductive layer 136 is arranged to surround the distal end of the groove.

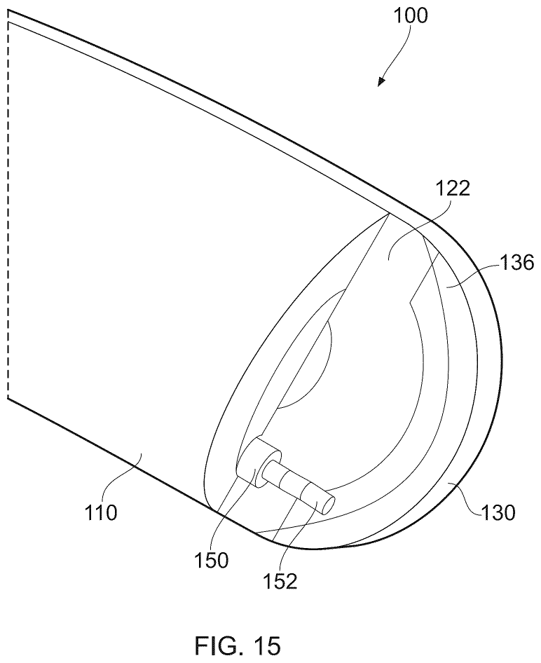

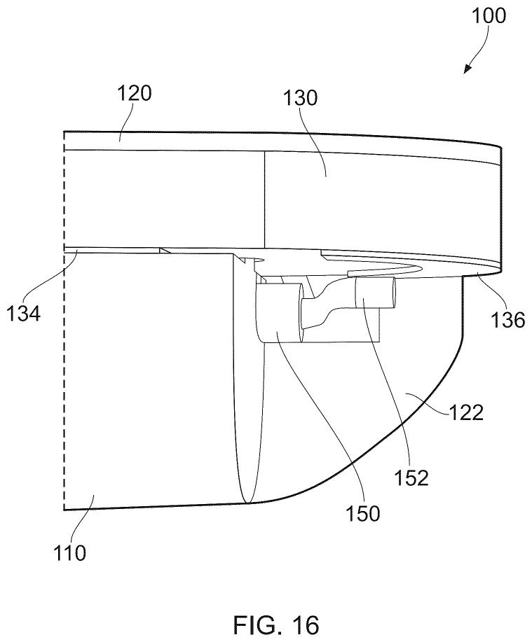

The active conductive layer 136, which, in this embodiment, is connected to the second transmission line via a feed cable 150, forms part of the second energy delivery structure. The feed cable 150 is connected to the active conductive layer 136 at a terminal 152. When assembled in the jaw 100, as illustrated in FIGS. 15 and 16, the active conductive layer 136 is arranged on a protruding lip (e.g. distally protruding lip) of the dielectric substrate 130 such that it can be placed in contact with biological tissue.

It is noted that the groove illustrated in the dielectric substrate 130 may be different from (e.g. larger, longer and/or wider than) the groove along which the blade 300 is slidable. In embodiments such as those illustrated in FIGS. 5 to 7, a portion of the conductive shell 110 may be locatable in the groove of the dielectric substrate, such that the groove 140 is contained within the conductive shell 110.

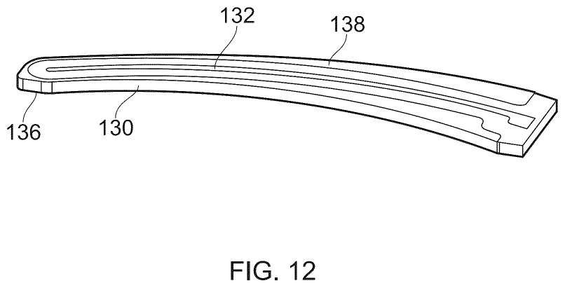

FIGS. 12, 13 and 14 illustrate two further embodiments of the power delivery structure of the first jaw 100. In these embodiments, the jaw 100 and the power delivery structure do not incorporate a groove or a blade. Furthermore, in these embodiments, the power delivery structure further incorporates a ground conductive strip 138 on a top surface of the dielectric substrate 130. The illustrated active conductive strip 132 is a finger electrode, and the ground conductive strip 138 flanks the active conductive strip 132 and surrounds its distal end. As with the embodiment illustrated in FIGS. 8 to 11, the energy delivery structure comprises the active conductive layer 136 at the distal end of the under surface of the dielectric substrate 130.

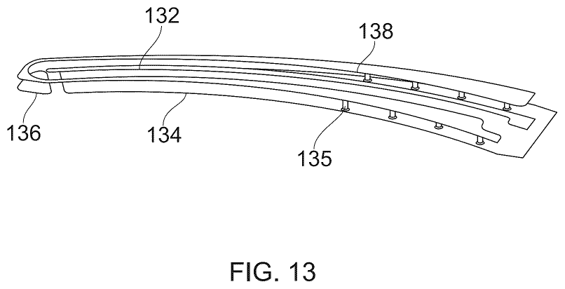

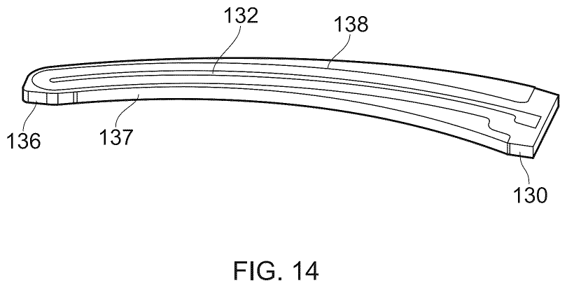

The ground conductive strip 138 may be connected to the ground conductive layer 134 via through holes 135 filled with conductive material which extend through the dielectric substrate 130. This is as illustrated in FIG. 13. In an embodiment, the through holes may be located only in a proximal portion of the jaw. The ground conductive strip 138 may be connected to the ground conductive layer via a conductive coating, strip, or layer 137 on an outer edge of the dielectric substrate 130. This is as illustrated in FIG. 14. In other embodiments, the conductive shell 110 may provide a connection between the ground conductive strip 138 and the ground conductive layer 134.

FIGS. 15 and 16 illustrate a distal end of the first jaw 100, with an arrangement that could utilise a power delivery structure as illustrated in FIGS. 8 to 11, or as illustrated in FIGS. 12 and 13, or as illustrated in FIG. 14. FIGS. 15 and 16 demonstrate the presence of the protruding lip of the dielectric substrate 130 on which the active conductive layer 136 is located. As will be understood, the active conductive layer 136 may extend proximally of the protruding lip, as may be required to be connected to a power feed cable 150 at a terminal 152. As shown, the power feed cable may be contained within an insulating sheath or tube to insulate it from other elements of the jaws. The active conductive layer 136 may cover all of the protruding lip, or only a part of the protruding lip as required to provide the particular tissue treatment effect wanted from the instrument.

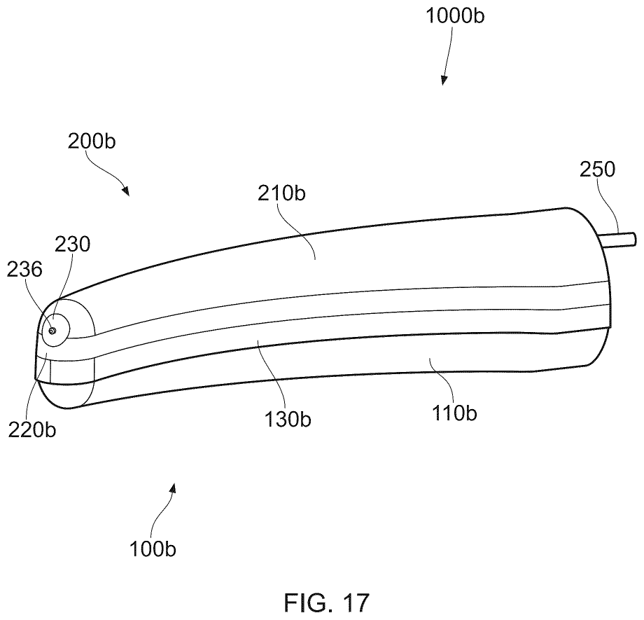

FIGS. 17 to 19 illustrates a distal assembly 1000b according to another aspect of the present invention. In this embodiment, a second energy delivery structure is located on and housed within the second jaw 200b, for example, on a distal end face of the second jaw 200b. The second energy delivery structure includes an active electrode 236 arranged on an outer surface of the second jaw 200b, and substantially flush with an outer surface of the conductive shell 210b of the second jaw 200b. The active electrode 236 is located within a dielectric plug 230, which isolates the electrode from the conductive shell 210b.

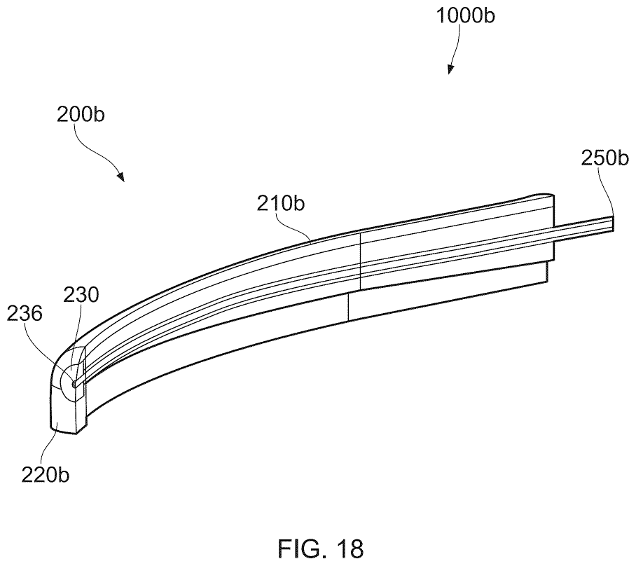

As illustrated in FIG. 18, the active electrode 236 is connected to a power supply via a feed cable 250, which is routed through the flexible body 220b of the second jaw 200b. In other arrangements, the cable 250 may instead be routed through (though insulated from) the conductive shell 210b. The conductive shell 210b or the conductive shell 110b may provide the return electrode for the second energy delivery structure. As shown, the power feed cable 250 may be contained within an insulating sheath or tube to insulate it from other elements of the jaws.

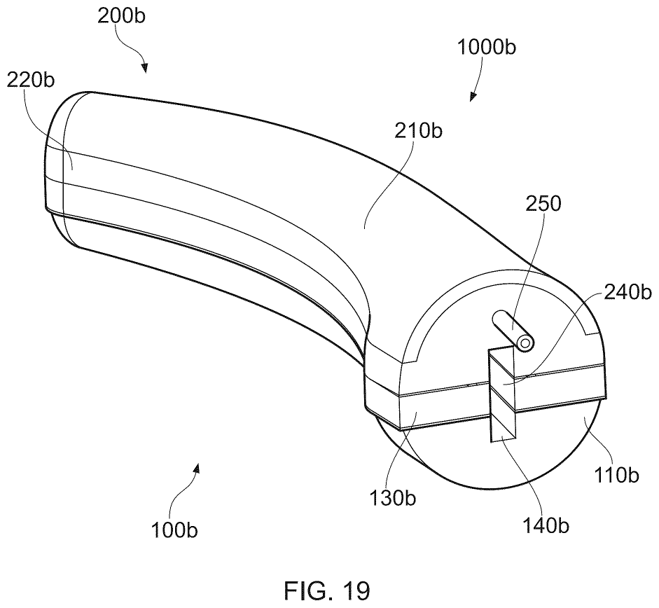

As with the earlier embodiments, and as shown in FIG. 19, the distal end assembly 1000b includes a groove 140b, 240b within each of the first jaw 100b and the second jaw 200b for receiving a blade.

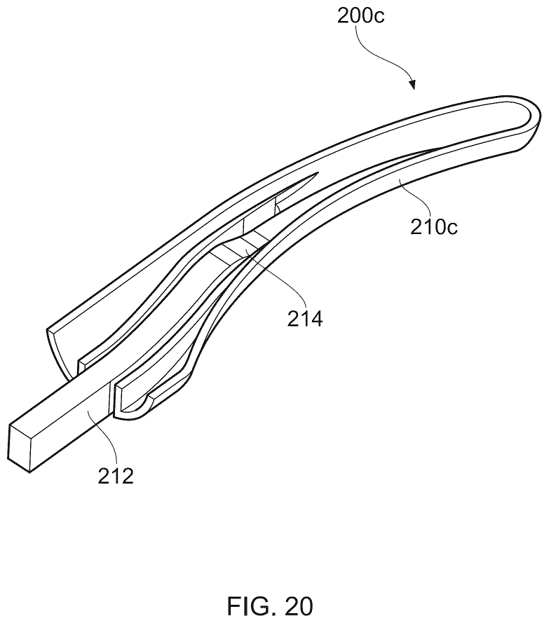

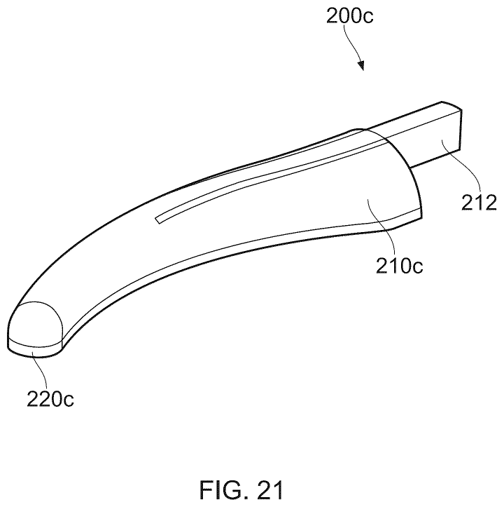

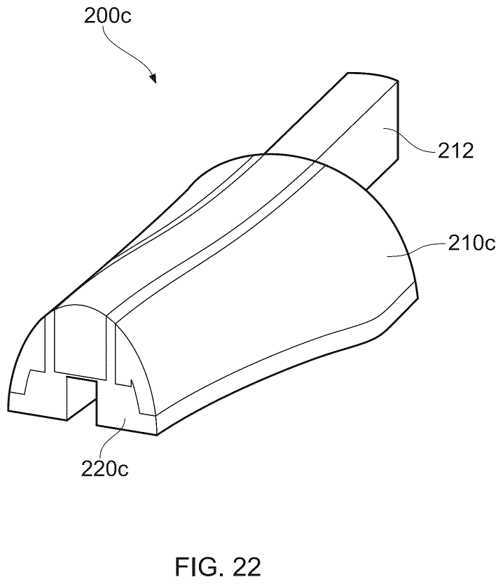

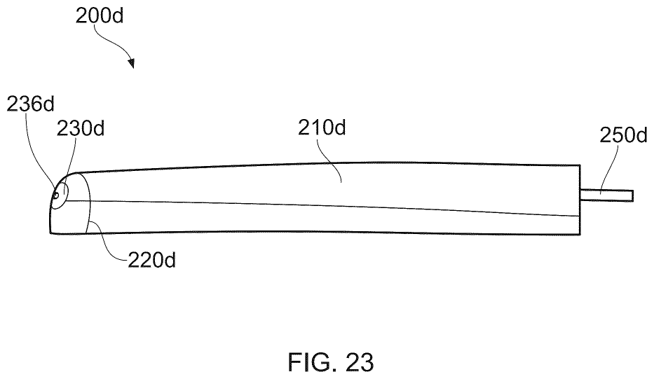

FIGS. 20 to 27 illustrate different embodiments for second jaws that can be used with the distal assemblies of the earlier embodiments. The embodiments of FIGS. 20 to 22 and 24 to 27 are also compatible with non-electrosurgical instruments (i.e. with surgical instruments with no energy delivery structure). Similarly, the embodiment of FIG. 23 would be compatible with non-electrosurgical instruments (i.e. with surgical instruments with no energy delivery structure) if the second energy delivery structure was omitted. Each of the second jaws 200c, 200d, 200e is configured to provide a more uniform pressure (clamping force or gripping force) along a length of the jaw when the jaws are closed (i.e. when the jaws 100, 200 are brought into contact).

The embodiment of the jaw 200c illustrated in FIGS. 20 to 22 includes a structure incorporating a rigid outer shell 210c and a strut 212 connected by a flexible hinge portion 214. In the illustrated embodiment, the outer shell 210c, strut 212 and hinge portion 214 are formed as a unitary component. The hinge portion 214 may be referred to as a living hinge. In other embodiments, the hinge portion 214 may be a flexible (e.g. bendable) portion of the strut 212 and may be unitary with only the strut 212, with the hinge portion 214 being bonded (e.g. welded) to the outer shell 210c. In further embodiments the hinge portion 214 may be a flexible (e.g. bendable) portion of the shell 210c and may be unitary with only the shell 210c, with the hinge portion 214 being bonded (e.g. welded) to the strut 212. In still further embodiments, the hinge portion 214 may be a flexible (e.g. bendable) strip or strut that is bonded (e.g. welded) to each of the strut 212 and the shell 210c to form the jaw.

The strut may be connected to a pivot point, such as a pivot point for the jaws of a distal end assembly. The hinge portion 214 may thus enable the rigid shell 210c to pivot at the hinge portion 214 with respect to the strut 212, i.e. at a second, different pivot point to the pivot point between the first and second jaws. This can enable the jaw 200c to provide a more even pressure along its length by enabling the jaw 200c to correct for unbalanced pressure. The jaw 200c of this embodiment further comprises a flexible body 220c which forms the inner surface of the second jaw 200c.

The jaw 200d illustrated in FIG. 23 comprises a flexible body 220d that increases in thickness (e.g. in a direction normal to a length of the jaw, or vertically as shown in FIG. 23) along a length of the second jaw 200d in a distal direction. In this way, a distal portion of the flexible body 220d is arranged to contact the inner surface of the first jaw 100 before a proximal portion of the flexible body 220d. The greater thickness of the flexible body 220d at the distal end provides a greater degree of compressibility, meaning that the thicker portion of the flexible body 220d can more readily adapt to relieve pressure on the clamped tissue at the distal end of the jaw 200d. For example, the flexible body 220d may first contact an inner surface of the first jaw 100 at a distal end of the first jaw 100, and may progressively deform along the flexible body 200d in a proximal direction of the jaws to provide a more even pressure along inner surfaces of the first 100 and second jaw 200d. In this way, the pressure is more evenly distributed along the length of the jaw 200d when the first jaw 100 and second jaw 200d are brought into contact. Thus the gripping action on tissue is improved.

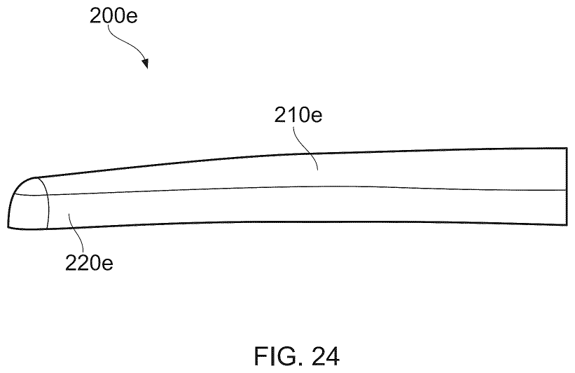

The jaw 200e illustrated in FIGS. 24 to 27 comprises a rigid shell 210e that is curved towards the opposite jaw along a length of the first jaw 100 in a distal direction, e.g. like the arm of a pair of tweezers. Thus, as with the embodiment illustrated in FIG. 23, the distal portion of the jaw 200e is configured to contact the first jaw of the pair of jaws before the proximal portion when the pair of jaws are moved towards each other and into contact.

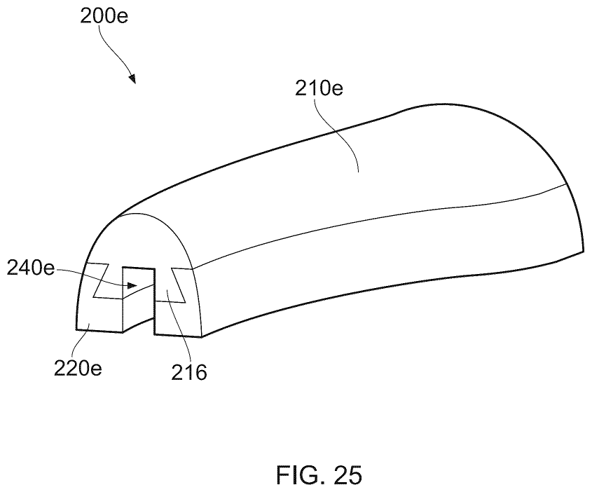

FIG. 25 illustrates a cross-section of the jaw 200e, illustrating a keyed or dovetail portion 216 of the rigid shell 210e for improved attachment to the flexible body 220e, which has a cooperating shaped groove for receiving and engaging (e.g. holding) the keyed portion 216. In other embodiments, this structure may be omitted.

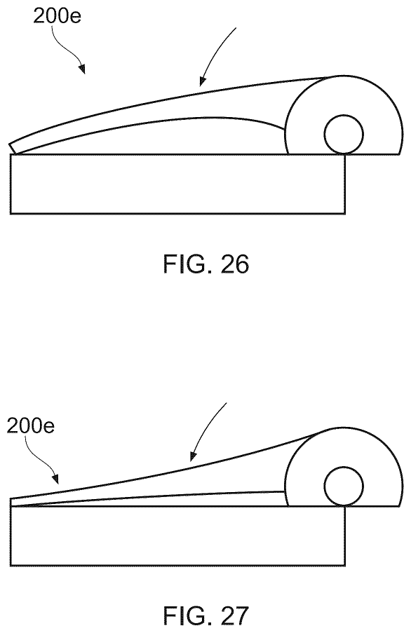

As illustrated in FIGS. 26 and 27, the rigid shell 210e may have a degree of flexibility, such that the shell 210e can flex or bend towards the first jaw 100 when the jaws are closed (see FIG. 27) providing improved distribution of the pressure along the jaws. For example, the shell may be made from a resilient or springy material, such as sprung steel or the like. In this way, the shell 210e of the upper jaw 200e may flex progressively in a proximal direction of the jaw 200e in a similar manner to an arm of a pair of tweezers. As the pressure is further increased, the entire upper jaw 200 may then compress evenly down onto the inner surface of the lower jaw 100 to provide the gripping force.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An electrosurgical instrument comprising:
an instrument shaft comprising a second transmission line for conveying radiofrequency (RF) EM energy;
a distal end assembly arranged at a distal end of the instrument shaft to receive the RF EM energy from the second transmission line, the distal end assembly comprising:
a pair of jaws that are movable relative to each other to open and close a gap between opposing inner surfaces thereof;
wherein a first jaw of the pair of jaws comprises:
a second energy delivery structure arranged to deliver the RF EM energy for cutting through biological tissue, the second energy delivery structure comprising:
a dielectric substrate having a top surface that is exposed at the gap between the opposing inner surfaces, and an under surface on an opposite side of the dielectric substrate from the top surface, the dielectric substrate protruding at a distal end of the first jaw to form an exposed lip on the under surface of the dielectric substrate at a distal end of the first jaw; and
a second active conductive layer on the under surface of the dielectric substrate, the second active conductive layer being arranged on the exposed lip.

2. The electrosurgical instrument according to claim 1, wherein:
the instrument shaft comprises a first transmission line for conveying microwave electromagnetic (EM) energy; and
wherein the first jaw further comprises a first energy delivery structure arranged to receive the microwave EM energy from the first transmission line and to emit the microwave EM energy into the gap between the opposing inner surfaces, the first energy delivery structure comprising:
a ground conductive layer on the under surface of the dielectric substrate, the ground conductive layer being electrically isolated from the second active conductive layer; and
an active conductive strip on the top surface of the dielectric substrate.

3. The electrosurgical instrument according to claim 2, wherein the first energy delivery structure further comprises a ground conductive strip on the top surface of the dielectric substrate and electrically connected to the ground conductive layer.

4. The electrosurgical instrument according to claim 3, wherein the ground conductive strip is electrically connected to the ground conductive layer via at least one of through holes formed in the dielectric substrate; and
a conductive strip on a side surface of the dielectric substrate.

5. The electrosurgical instrument according to claim 2, wherein the first jaw comprises an electrically conductive outer shell, the outer shell being electrically connected to the ground conductive layer.

6. The electrosurgical instrument according to claim 5, wherein the ground conductive strip is electrically connected to the ground conductive layer via the electrically conductive outer shell.

7. An electrosurgical instrument comprising:
an instrument shaft comprising a first transmission line for conveying microwave electromagnetic (EM) energy;
a distal end assembly arranged at a distal end of the instrument shaft to receive the microwave EM energy from the first transmission line, the distal end assembly comprising:
a pair of jaws that are movable relative to each other to open and close a gap between opposing inner surfaces thereof;
wherein a first jaw of the pair of jaws comprises:
an energy delivery structure arranged to emit the microwave EM energy into the gap between the opposing inner surfaces, the energy delivery structure comprising:
a dielectric substrate having a top surface that is exposed at the gap between the opposing inner surfaces, and an under surface on an opposite side of the dielectric substrate from the top surface;
a ground conductive layer on the under surface; and
an active conductive strip on the top surface; and
wherein the first jaw comprises an electrically conductive outer shell, the outer shell being electrically connected to the ground conductive layer.

8. The electrosurgical instrument according to claim 7, wherein the first jaw comprises a longitudinally extending recessed groove on the inner surface of the first jaw for receiving a blade for cutting through biological tissue.

9. The electrosurgical instrument according to claim 8, wherein the dielectric substrate is arranged on a top surface of the electrically conductive outer shell, the top surface of the electrically conductive outer shell being a surface of the electrically conductive outer shell closest to the inner surface of the first jaw, and wherein the recessed groove for receiving the blade extends from the inner surface of the jaw through the dielectric substrate and into the electrically conductive shell.

10. The electrosurgical instrument according to claim 8, wherein the dielectric substrate is received in a second recessed groove on a top surface of the electrically conductive outer shell, the top surface of the electrically conductive outer shell being a surface of the electrically conductive outer shell closest to the inner surface of the first jaw, and wherein the recessed groove for receiving the blade extends from the inner surface of the jaw into the electrically conductive shell and adjacent to the recessed groove for receiving the blade.

11. The electrosurgical instrument according to claim 10, wherein the second recessed groove is open to a side surface of the first jaw, such that the dielectric substrate is exposed at the side surface of the jaw.

12. The electrosurgical instrument according to claim 10, wherein the second recessed groove is within the electrically conductive shell.

13. The electrosurgical instrument according to claim 10, wherein the second recessed groove is a U-shaped groove that flanks the recessed groove for receiving the blade and surrounds its distal end.

14. The electrosurgical instrument according to claim 8, further comprising a blade for cutting through biological tissue, the blade being slidably mounted within the recessed groove, and wherein the blade is:
slidable in a longitudinal direction between a retracted position in which the blade is stowed and an extended position for cutting tissue in the gap; or
slidable in a lateral direction between a retracted position in which it lies beneath the inner surface of the first jaw and an extended position in which it lies within a region between the pair of jaws.

15. The electrosurgical instrument according to claim 14, wherein the blade comprises a rigid element with a sharp edge adapted to slice biological tissue.

16. The electrosurgical instrument according to claim 7, wherein the instrument shaft comprises a second transmission line for conveying radiofrequency (RF) EM energy; and
wherein the pair of jaws comprises a second energy delivery structure arranged to deliver the RF EM energy for cutting through biological tissue, the second energy delivery structure comprising:
a second active conductive layer on the under surface of the dielectric substrate, the second active conductive layer being arranged on an exposed lip at a distal end of the first jaw and being electrically isolated from the ground conductive layer.

17. An electrosurgical instrument comprising:
an instrument shaft arranged to convey radiofrequency (RF) EM energy;
a distal end assembly arranged at a distal end of the instrument shaft to receive the RF EM energy from the instrument shaft, the distal end assembly comprising:
a pair of jaws that are movable relative to each other to open and close a gap between opposing inner surfaces thereof;
wherein a second jaw of the pair of jaws comprises:
an electrically conductive outer shell; and
an active electrode arranged on an outer surface of the jaw, the active electrode being for delivering RF energy to biological tissue, the active electrode being electrically isolated from the electrically conductive outer shell,
wherein the electrically conductive outer shell is grounded to form a return electrode for the RF energy, and
wherein the active electrode is substantially flush with the outer surface of the jaw.

18. The electrosurgical instrument according to claim 17, wherein the active electrode is comprised within a dielectric plug, the dielectric plug forming part of the outer surface of the jaw and being arranged to electrically isolate the active electrode from the electrically conductive outer shell.

19-25. (canceled)
